SEQUENCE LISTING (15 sequences)

SEQ ID NO: 1 - DNA, 798 bp, Artificial Sequence
  Chemically synthesized, HPV genotype 16 E6-E7 DNA sequence
atggactggacctggatcctgttcctggtggccgccgccacacgggtgcacagcttccaggacccccaggagagcggcagaaagctgcctcagctgtgtaccgagctgcagaccaccatccacgacatcatcctggagtgtgtgtactgtaagcagcagctgctgaggagagaggtgtacgaccgggacctgtgtatcgtgtacagggacggcaatccctacgccgtgtgtgacaagtgcctgaagttctacagcaagatcagcgagtaccggcactactgctacagcctgtacggcaccaccctggagcagcagtacaacaagcccctgtgtgacctgctgatccggtgtatcaactgccagaagcccctgcagagacacctggacaagaagcagcggttccacaacatcaggggcagatggaccggcagatgtatgagctgctgccggagcagcagaaccagaagggagacccagctgagaggccggaagagaagaagccacggcgatacccccaccctgcacgagtacatgctggacctgcagcctgagaccaccgatctgtacggctacggccagctgaatgacagcagcgaggaggaggatgagatcgacggccctgccggccaggccgagcccgacagagcccactacaacatcgtgaccttttgctgtaagtgtgacagcaccctgagactgtgcgtgcagagcacccacgtggacatcagaaccctggaggatctgctgatgggcaccctgggcatcgtgtgtcccatctgctcccagaaaccctgatga

SEQ ID NO: 2 - protein, 264 aa, Artificial Sequence
  Chemically synthesized, HPV genotype 16 E6-E7 protein sequence
MDWTWILFLVAAATRVHSFQDPQESGRKLPQLCTELQTTIHDIILECVYCKQQLLRREVYDRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQLRGRKRRSHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 3 - protein, 15 aa, Artificial Sequence
  Chemically synthesized, HPV16 E6 immunodominant epitope
LCIVYRDGNPYAVCD

SEQ ID NO: 4 - protein, 15 aa, Artificial Sequence
  Chemically synthesized, HPV16 E7 immunodominant epitope
AEPDRAHYNIVTFCC

SEQ ID NO: 5 - protein, 142 aa, Artificial Sequence
  Chemically synthesized, HPV16 E6 consensus sequence
FQDPQESGRKLPQLCTELQTTIHDIILECVYCKQQLLRREVYDRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQL

SEQ ID NO: 6 - protein, 97 aa, Artificial Sequence
  Chemically synthesized, HPV16 E7 consensus sequence
HGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 7 - protein, 18 aa, Artificial Sequence
  Chemically synthesized, IgE Leader Sequence
MDWTWILFLVAAATRVHS

SEQ ID NO: 8 - protein, 7 aa, Artificial Sequence
  Chemically synthesized, Proteolytic Cleavage Sequence
RGRKRRS

SEQ ID NO: 9 - DNA, 783 bp, Artificial Sequence
  Chemically synthesized, HPV18 Consensus Sequence encoding E6 and E7
gccagattcgaggaccccaccaggagcggctacaagctgcccgatctgtgtaccgagctgaacaccagcctgcaggacatcgagatcacctgtgtgtactgtaagaccgtgctggagctgaccgaggtgttcgagaaggacctgttcgtggtgtacagggacagcatcccccacgccgcctgccacaagtgtatcgacttctacagccggatccgggagctgagacactacagcgacagcgtgtacggcgataccctggagaagctgaccaacaccggcctgtacaacctgctgatccggtgcctgagatgccagaagcccctgctgagacacctgaacgagaagcggcggttccacaacatcgccggccactacagaggccagtgccacagctgctgtaacagggccaggcaggagagactgcagcggagaagagagacccaggtgaggggcaggaagagaagaagccacggccccaaggccaccctgcaggatatcgtgctgcacctggagccccagaatgagatccccgtggatctgctgggccacggccagctgtccgacagcgaggaggagaacgacgagatcgacggcgtgaatcaccagcacctgcctgccagaagagccgagcctcagaggcacaccatgctgtgtatgtgctgtaagtgtgaggcccggatcgaactggtggtggagagcagcgccgacgacctgagagccttccagcagctgttcctgaacaccctgagcttcgtgtgtccttggtgtgccagccagcagtga

SEQ ID NO: 10 - protein, 260 aa, Artificial Sequence
  Chemically synthesized, HPV18 Consensus HPV peptide sequence with E6 and E7
ARFEDPTRSGYKLPDLCTELNTSLQDIEITCVYCKTVLELTEVFEKDLFVVYRDSIPHAACHKCIDFYSRIRELRHYSDSVYGDTLEKLTNTGLYNLLIRCLRCQKPLLRHLNEKRRFHNIAGHYRGQCHSCCNRARQERLQRRRETQVRGRKRRSHGPKATLQDIVLHLEPQNEIPVDLLGHGQLSDSEEENDEIDGVNHQHLPARRAEPQRHTMLCMCCKCEARIELVVESSADDLRAFQQLFLNTLSFVCPWCASQQ

SEQ ID NO: 11 - DNA, 54 bp, Artificial Sequence
  Chemically synthesized, IgE Leader DNA sequence
atggactggacctggatcctgttcctggtggccgctgccacacgggtgcacagc

SEQ ID NO: 12 - protein, 18 aa, Artificial Sequence
  Chemically synthesized, IgE leader peptide sequence
MDWTWILFLVAAATRVHS

SEQ ID NO: 13 - DNA, 837 bp, Artificial Sequence
  Chemically synthesized, HPV18 Consensus Sequence encoding E6 and E7 include IgE leader
atggactggacctggatcctgttcctggtggccgctgccacacgggtgcacagcgccagattcgaggaccccaccaggagcggctacaagctgcccgatctgtgtaccgagctgaacaccagcctgcaggacatcgagatcacctgtgtgtactgtaagaccgtgctggagctgaccgaggtgttcgagaaggacctgttcgtggtgtacagggacagcatcccccacgccgcctgccacaagtgtatcgacttctacagccggatccgggagctgagacactacagcgacagcgtgtacggcgataccctggagaagctgaccaacaccggcctgtacaacctgctgatccggtgcctgagatgccagaagcccctgctgagacacctgaacgagaagcggcggttccacaacatcgccggccactacagaggccagtgccacagctgctgtaacagggccaggcaggagagactgcagcggagaagagagacccaggtgaggggcaggaagagaagaagccacggccccaaggccaccctgcaggatatcgtgctgcacctggagccccagaatgagatccccgtggatctgctgggccacggccagctgtccgacagcgaggaggagaacgacgagatcgacggcgtgaatcaccagcacctgcctgccagaagagccgagcctcagaggcacaccatgctgtgtatgtgctgtaagtgtgaggcccggatcgaactggtggtggagagcagcgccgacgacctgagagccttccagcagctgttcctgaacaccctgagcttcgtgtgtccttggtgtgccagccagcagtga

SEQ ID NO: 14 - protein, 278 aa, Artificial Sequence
  Chemically synthesized, HPV18 Consensus HPV peptide sequence with E6 and E7 including IgE leader
MDWTWILFLVAAATRVHSARFEDPTRSGYKLPDLCTELNTSLQDIEITCVYCKTVLELTEVFEKDLFVVYRDSIPHAACHKCIDFYSRIRELRHYSDSVYGDTLEKLTNTGLYNLLIRCLRCQKPLLRHLNEKRRFHNIAGHYRGQCHSCCNRARQERLQRRRETQVRGRKRRSHGPKATLQDIVLHLEPQNEIPVDLLGHGQLSDSEEENDEIDGVNHQHLPARRAEPQRHTMLCMCCKCEARIELVVESSADDLRAFQQLFLNTLSFVCPWCASQQ

SEQ ID NO: 15 - DNA, 3824 bp, Artificial Sequence
  Chemically synthesized, pGX3002 plasmid with consensus and IgE leader
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatggactggacctggatcctgttcctggtggccgctgccacacgggtgcacagcgccagattcgaggaccccaccaggagcggctacaagctgcccgatctgtgtaccgagctgaacaccagcctgcaggacatcgagatcacctgtgtgtactgtaagaccgtgctggagctgaccgaggtgttcgagaaggacctgttcgtggtgtacagggacagcatcccccacgccgcctgccacaagtgtatcgacttctacagccggatccgggagctgagacactacagcgacagcgtgtacggcgataccctggagaagctgaccaacaccggcctgtacaacctgctgatccggtgcctgagatgccagaagcccctgctgagacacctgaacgagaagcggcggttccacaacatcgccggccactacagaggccagtgccacagctgctgtaacagggccaggcaggagagactgcagcggagaagagagacccaggtgaggggcaggaagagaagaagccacggccccaaggccaccctgcaggatatcgtgctgcacctggagccccagaatgagatccccgtggatctgctgggccacggccagctgtccgacagcgaggaggagaacgacgagatcgacggcgtgaatcaccagcacctgcctgccagaagagccgagcctcagaggcacaccatgctgtgtatgtgctgtaagtgtgaggcccggatcgaactggtggtggagagcagcgccgacgacctgagagccttccagcagctgttcctgaacaccctgagcttcgtgtgtccttggtgtgccagccagcagtgatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

(19) United States
(12) Patent Application Publication
(10) Pub. No.: US 2023/0277645 A1
(43) Pub. Date: Sep. 7, 2023

(54) COMPOSITIONS AND METHODS FOR TREATING VULVAR DYSPLASIA

(71) Applicant: Inovio Pharmaceuticals, Inc., Plymouth Meeting, PA (US)

(72) Inventor: Jian Yan, Wallingford, PA (US)

(21) Appl. No.: 17/221,032

(22) Filed: Apr. 2, 2021

Related U.S. Application Data
(60) Provisional application No. 63/168,173, filed on Mar. 30, 2021.
(60) Provisional application No. 63/004,161, filed on Apr. 2, 2020.

(51) Int. Cl. A61K 39/12; A61K 41/00

(52) U.S. Cl. CPC A61K 39/12; A61K 41/0047; A61K 2039/54

(57) ABSTRACT

The use of anti-HPV immunogens and nucleic acid molecules that encode them for the treatment and prevention of vulvar dysplasia are disclosed. Pharmaceutical composition, recombinant vaccines comprising DNA plasmid and live attenuated vaccines are disclosed as well methods of inducing an immune response to treat or prevent vulvar dysplasia are disclosed.

### CROSS-REFERENCE TO RELATED APPLICATIONS

The present application claims the benefit of U.S. Provisional Appl. No. 63/004,161 filed Apr. 2, 2020 and U.S. Appl. No. 63/168,173 filed Mar. 30, 2021. The contents of each of these applications are incorporated herein by reference in their entirety.

### SEQUENCE LISTING

This application contains a Sequence Listing which has been submitted electronically in ASCII format and is hereby incorporated by reference in its entirety. Said ASCII copy, created May 21, 2021, is named INO-3100-US-Sequence-Listing.txt and is 20,608 bytes in size.

### FIELD OF THE INVENTION

The present invention relates to improved vaccines, improved methods for inducing immune responses, and for prophylactically and/or therapeutically immunizing individuals against vulvar dysplasia.

### BACKGROUND OF THE INVENTION

The current management of vulvar dysplasia, also referred to as vulvar high grade squamous intraepithelial lesions (HSIL), is challenging. Persistent infection with one or more high-risk HPV genotypes can lead to the development of precancerous, histologic HSIL. Human papillomaviruses of subtype 16 and 18 are considered high-risk, and are involved in approximately 83 percent of HPV-related vulvar HSIL cases in the United States.

The literature suggests that less than 1 in 20 women with vulvar HSIL exhibit a spontaneous regression of their lesion. Surgical treatments such as excision, vulvectomy, laser ablation, and off-label medical therapy are not only disfiguring, but also have a significant risk of reoccurrence, with sources citing up to 34 percent reoccurrence at 6 months and 45 percent reoccurrence three years post-treatment.

Thus, there is a need in the art for improved compositions and methods for treatment or prevention of vulvar dysplasia. The present invention satisfies this unmet need.

### SUMMARY OF THE INVENTION

Aspects of the invention provide compositions comprising at least one nucleotide sequence comprising an HPV16 E6-E7 fusion antigen, an HPV18 E6-E7 fusion antigen, or a combination thereof, and uses thereof for the treatment or prevention of vulvar dysplasia.

Another aspect provides compositions comprising one or more nucleotide sequences encoding an HPV16 E6-E7 fusion antigen selected from the group consisting of: nucleotide sequence that encodes SEQ ID NO:2; a nucleotide sequence that is at least 95% homologous to a nucleotide sequence that encodes SEQ ID NO:2; a nucleotide sequence that is at least 95% homologous to a fragment of a nucleotide sequence that encodes SEQ ID NO:2. In some embodiments, the nucleotide sequences encoding the HPV6 E6-E7 fusion antigen are without a leader sequence at 5′ end.

In another aspect of the invention, there are provided compositions comprising one or more nucleotide sequences encoding an HPV16 E6-E7 fusion antigen selected from the group consisting of: SEQ ID NO:1; a nucleotide sequence that is at least 95% homologous to SEQ ID NO:1; a fragment of SEQ ID NO:1; a nucleotide sequence that is at least 95% homologous to a fragment of SEQ ID NO:1. In some embodiments, the nucleotide sequences encoding the HPV16 E6-E7 fusion antigen are without a leader sequence at 5′ end.

Another aspect provides compositions comprising one or more nucleotide sequences encoding an HPV18 E6-E7 fusion antigen selected from the group consisting of: nucleotide sequence that encodes SEQ ID NO:10; a nucleotide sequence that is at least 95% homologous to a nucleotide sequence that encodes SEQ ID NO:10; a nucleotide sequence that is at least 95% homologous to a fragment of a nucleotide sequence that encodes SEQ ID NO:10. In some embodiments, the nucleotide sequences encoding the HPV6 E6-E7 fusion antigen are further comprises a nucleotide encoding a leader sequence at the 5′ end.

In another aspect of the invention, there are provided compositions comprising one or more nucleotide sequences encoding an HPV18 E6-E7 fusion antigen selected from the group consisting of: SEQ ID NO:9; a nucleotide sequence that is at least 95% homologous to SEQ ID NO:9; a fragment of SEQ ID NO:9; a nucleotide sequence that is at least 95% homologous to a fragment of SEQ ID NO:9. In some embodiments, the nucleotide sequences encoding the HPV16 E6-E7 fusion antigen further comprises a nucleotide encoding a leader sequence at the 5′ end.

The nucleotide sequences provided can be a plasmid.

In additional aspects, provided are pharmaceutical compositions comprising the disclosed nucleotide sequences.

In some aspects, there are methods of treating or preventing vulvar dysplasia in an individual by inducing an effective immune response in an individual, comprising administering to said individual a composition comprising one or more of the nucleotides sequences provided. The methods preferably include a step of introducing the provided nucleotide sequences into the individual by electroporation.

### BRIEF DESCRIPTION OF THE FIGURE

The patent or application file contains at least one drawing executed in color. Copies of this patent or patent application publication with color drawing(s) will be provided by the Office upon request and payment of the necessary fee.

FIG. 1 depicts the study design of the present study.

FIG. 2 depicts the enrollment status of subjects in the present study.

FIG. 3 depicts the demographics and other baseline data for the subjects in the VGX-3100 treatment group (n=25).

FIG. 4 depicts the safety events for the VGX-3100 group.

FIG. 5 depicts the number of subjects with confirmed HSIL and non-HPV16/18 types at screening at week 48 in a subset of the VGX-3100 group.

FIG. 6 depicts the efficacy assessment at week 48 for the VGX-3100 group (n=20; results from 4 subjects were not evaluable.)

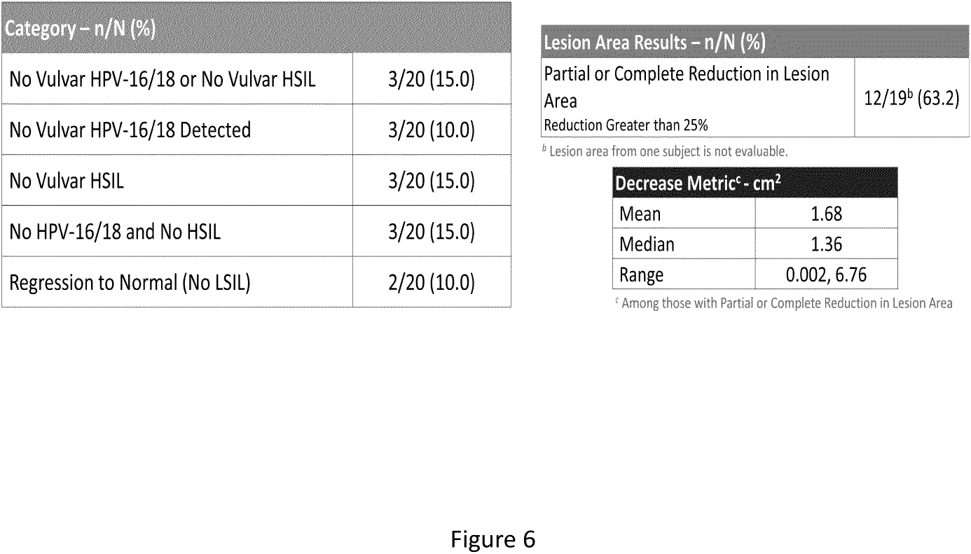

### DETAILED DESCRIPTION OF PREFERRED EMBODIMENTS

### Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### A. Adjuvant

“Adjuvant” as used herein may mean any molecule added to the DNA plasmid vaccines described herein to enhance antigenicity of the one or more antigens encoded by the DNA plasmids and encoding nucleic acid sequences described hereinafter.

### B. Antibody

“Antibody” may mean an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

### C. Antigen

“Antigen” refers to: proteins having an HPV E6 or HPV E7 domain, and preferably and E6 and E7 fusion with an endeoproteolytic cleavage site therebetween. Antigens include SEQ ID NO: 2 (subtype 16) and SEQ ID NO: 4 (subtype 18); fragments thereof of lengths set forth herein, variants, i.e. proteins with sequences homologous to SEQ ID NO:2 or SEQ ID NO:4 as set forth herein, fragments of variants having lengths set forth herein, and combinations thereof. Antigens may have an IgE leader sequence of SEQ ID NO:7 or 12 or may alternatively have such sequence removed from the N-terminal end. Antigens may optionally include signal peptides such as those from other proteins.

### D. Coding Sequence

“Coding sequence” or “encoding nucleic acid” as used herein may mean refers to the nucleic acid (RNA or DNA molecule) that comprise a nucleotide sequence which encodes an antigen as set forth in section c. above. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides, e.g., an IgE leader sequence such as SEQ ID NO:7 or 12.

### E. Complement

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

### F. Fragment

“Fragment” may mean a polypeptide fragment of an antigen that is capable of eliciting an immune response in a mammal against the antigen. A fragment of an antigen may be 100% identical to the full length except missing at least one amino acid from the N and/or C terminal, in each case with or without signal peptides and/or a methionine at position 1. Fragments may comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antigen, excluding any heterologous signal peptide added. The fragment may, preferably, comprise a fragment of a polypeptide that is 95% or more, 96% or more, 97% or more, 98% or more or 99% or more homologous to the antigen and additionally comprise an N terminal methionine or heterologous signal peptide which is not included when calculating percent homology Fragments may further comprise an N terminal methionine and/or a signal peptide such as an immunoglobulin signal peptide, for example an IgE or IgG signal peptide. The N terminal methionine and/or signal peptide may be linked to a fragment of an antigen.

A fragment of a nucleic acid sequence that encodes antigen may be 100% identical to the full length except missing at least one nucleotide from the 5′ and/or 3′ end, in each case with or without sequences encoding signal peptides and/or a methionine at position 1. Fragments may comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may, preferably, comprise a fragment that encodes a polypeptide that is 95% or more, 96% or more, 97% or more, 98% or more or 99% or more homologous to the antigen and additionally optionally comprise sequence encoding an N terminal methionine or heterologous signal peptide which is not included when calculating percent homology Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

### G. Identical

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

### H. Immune Response

“Immune response” as used herein may mean the activation of a host’s immune system, e.g., that of a mammal, in response to the introduction of one or more antigens via the provided DNA plasmid vaccines. The immune response can be in the form of a cellular or humoral response, or both.

### I. Nucleic Acid

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

### J . Operably Linked

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

### K. Promoter

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

### 1. Stringent Hybridization Conditions

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 510° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5x SSC, and 1% SDS, incubating at 42° C., or, 5x SSC, 1% SDS, incubating at 65° C., with wash in 0.2x SSC, and 0.1% SDS at 65° C.

### M. Substantially Complementary

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

### N. Substantially Identical

“Substantially identical” as used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

### O. Variant

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

### P. Vector

“Vector” used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

### Description

Improved vaccines are disclosed which arise from a multi-phase strategy to enhance cellular immune responses induced by immunogens. Modified consensus sequences were generated. Genetic modifications including codon optimization, RNA optimization, and the addition of a high efficient immunoglobin leader sequence are also disclosed. The novel construct has been designed to elicit stronger and broader cellular immune responses than a corresponding codon optimized immunogens.

The improved HPV vaccines are based upon proteins and genetic constructs that encode proteins with epitopes that make them particularly effective as immunogens, such that they mediate a prophylactic or therapeutic strategy against vulvar dysplasia, also referred to as vulvar high grade squamous intraepithelial lesions (HSIL). Accordingly, vaccines may induce a therapeutic or prophylactic immune response. In some embodiments, the means to deliver the immunogen is a DNA vaccine, a recombinant vaccine, a protein subunit vaccine, a composition comprising the immunogen, an attenuated vaccine or a killed vaccine. In some embodiments, the vaccine comprises a combination selected from the groups consisting of: one or more DNA vaccines, one or more recombinant vaccines, one or more protein subunit vaccines, one or more compositions comprising the immunogen, one or more attenuated vaccines and one or more killed vaccines.

According to some embodiments, a vaccine is delivered to an individual to modulate the activity of the individual’s immune system and thereby enhance the immune response against HPV to treat vulvar dysplasia. When a nucleic acid molecule that encodes the protein is taken up by cells of the individual the nucleotide sequence is expressed in the cells and the protein are thereby delivered to the individual. Methods of delivering the coding sequences of the protein on nucleic acid molecule such as plasmid, as part of recombinant vaccines and as part of attenuated vaccines, as isolated proteins or proteins part of a vector are provided.

Compositions and methods are provided which provide a prophylactic and/or therapeutic treatment against vulvar dysplasia in an individual.

Compositions for delivering nucleic acid molecules that comprise a nucleotide sequence that encodes the immunogen are operably linked to regulatory elements. Compositions may include a plasmid that encodes the immunogen, a recombinant vaccine comprising a nucleotide sequence that encodes the immunogen, a live attenuated pathogen that encodes a protein of the invention and/or includes a protein of the invention; a killed pathogen includes a protein of the invention; or a composition such as a liposome or subunit vaccine that comprises a protein of the invention. The present invention further relates to injectable pharmaceutical compositions that comprise compositions.

Aspects of the invention provide compositions comprising at least one nucleotide sequence encoding at least one HPV E6-E7 fusion antigen, for example an HPV16 E6-E7 fusion antigen or an HPV18 E6-E7 fusion antigen. In one embodiment, the composition comprises a nucleotide sequence encoding an HPV16 E6-E7 fusion antigen and an HPV18 E6-E7 fusion antigen.

In one embodiment, the invention include methods of administrating the composition of the invention into a subject in need thereof. In one embodiment, the subject is a subject diagnosed with vulvar dysplasia. In one embodiment, the subject is subject having vulvar dysplasia. In one embodiment, the subject is a subject at risk of developing vulvar dysplasia.

### HPV16 E6-E7 Fusion

Another aspect provides compositions comprising one or more nucleotide sequences encoding an HPV16 E6-E7 fusion antigen selected from the group consisting of: nucleotide sequence that encodes SEQ ID NO:2; a nucleotide sequence that is at least 95% homologous to a nucleotide sequence that encodes SEQ ID NO:2; a fragment of a nucleotide sequence that encodes SEQ ID NO:2; a nucleotide sequence that is at least 95% homologous to a fragment of a nucleotide sequence that encodes SEQ ID NO:2.

In some embodiments the compositions include HPV16 E6-E7 fusion antigens selected from the group consisting of: nucleotide sequence that encodes SEQ ID NO:2; a nucleotide sequence that is at least 95% homologous to a nucleotide sequence that encodes SEQ ID NO:2; a fragment of a nucleotide sequence that encodes SEQ ID NO:2; a nucleotide sequence that is at least 95% homologous to a fragment of a nucleotide sequence that encodes SEQ ID NO:2.

In another aspect of the invention, there are provided compositions comprising one or more nucleotide sequences encoding an HPV16 E6-E7 fusion antigen selected from the group consisting of: SEQ ID NO:1; a nucleotide sequence that is at least 95% homologous to SEQ ID NO:1; a fragment of SEQ ID NO:1; a nucleotide sequence that is at least 95% homologous to a fragment of SEQ ID NO:1.

In some embodiments the nucleotide sequences described herein is absent the leader sequence. In one embodiment, the nucleotide sequences comprising HPV16 E6-E7 fusion antigen is absent a leader sequence. In particular, the HPV16 E6-E7 fusion antigens including nucleotide sequence that encodes SEQ ID NO:2; are absent a leader sequence at 5′ end, for example nucleotide sequence encoding SEQ ID NO:7. In particular, the HPV6 E6-E7 fusion antigens including nucleotide sequence SEQ ID NO:1 are absent a leader sequence at 5′ end, for example nucleotide sequence encoding SEQ ID NO:7.

In some embodiments the nucleotide sequences of the present invention can be 80%, 81%, 82%, 83%, 84%, 85%, 86%, 87%, 88%, 89%, 90%, 91%, 92%, 93%, 94%, 95%, 96%, 97%, 98%, or 99% homologous with the provided nucleotide sequences; preferably 95%, 96%, 97%, 98%, or 99%; or 98% or 99%.

The nucleotide sequences provided can be included into one of a variety of known vectors or delivery systems, including a plasmid, viral vector, lipid vector, nanoparticle.; preferably a plasmid.

In additional aspects, provided are pharmaceutical compositions comprising the disclosed nucleotide sequences.

In some aspects, there are methods of inducing an effective immune response in an individual against more than one subtype of HPV thereby providing a prophylactic or therapeutic treatment against vulvar dysplasia, comprising administering to said individual a composition comprising one or more of the nucleotides sequences provided; preferably, the compositions have more than one antigen. The methods preferably include a step of introducing the provided nucleotide sequences into the individual by electroporation.

SEQ ID NO:1 comprises a nucleotide sequence that encodes a consensus immunogen of HPV16 E6 and E7 proteins, that comprises and IgE leader sequence, a consensus sequence for HPV E6, linked to a consensus sequence for HPV E7 by a proteolytic cleavage sequence. SEQ ID NO: 2 comprises the amino acid sequence of a consensus immunogen of HPV16 E6 and E7 proteins, that comprises and IgE leader sequence, a consensus sequence for HPV E6, linked to a consensus sequence for HPV E7 by a proteolytic cleavage sequence. The consensus sequence for HPV16 E6 includes the immunodominant epitope set forth in SEQ ID NO:3. The consensus sequence for HPV16 E7 includes the immunodominant epitope set forth in SEQ ID NO:4. The consensus sequence for HPV E6 is SEQ ID NO:5. The consensus sequence for HPV E6 is SEQ ID NO:6. The IgE leader sequence is SEQ ID NO:7. A proteolytic cleavage sequence useful to link the two consensus sequences is SEQ ID NO:8.

Further information regarding the HPV16 E6-E7 fusion antigen can be found at least in U.S. Pat. No. 8,168,769, which is incorporated by reference in its entirety.

In some embodiments, vaccines include SEQ ID NO:2, or a nucleic acid molecule that encodes SEQ ID NO:2. In some embodiments, vaccines of the invention include SEQ ID NO:3 and/or SEQ ID NO:4, or nucleic acid sequence which encode one of both of them. In some embodiments, vaccines of the invention include SEQ ID NO:5 and/or the SEQ ID NO:6, or nucleic acid sequences which encode one or both of them. In some embodiments, vaccines of the invention include SEQ ID NO:5 linked to SEQ ID NO:6 by a proteolytic cleavage sequence such as SEQ ID NO:8, or nucleic acid sequence which encodes the fusion protein. In some embodiments, vaccines of the present invention include the IgE leader sequence SEQ ID NO:7 or nucleic acid sequence which encodes the same. In some embodiments, vaccines of the invention include SEQ ID NO:2 or the nucleic acid sequence in SEQ ID NO:1.

Fragments of SEQ ID NO:2 may be 100% identical to the full length except missing at least one amino acid from the N and/or C terminal, in each case with or without signal peptides and/or a methionine at position 1. Fragments of SEQ ID NO:2 can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the full length SEQ ID NO:2, excluding any heterologous signal peptide added. The fragment can, preferably, comprise a fragment of SEQ ID NO:2 that is 95% or more, 96% or more, 97% or more, 98% or more or 99% or more homologous to SEQ ID NO:2 and additionally comprise an N terminal methionine or heterologous signal peptide which is not included when calculating percent homology Fragments can further comprise an N terminal methionine and/or a signal peptide such as an immunoglobulin signal peptide, for example an IgE or IgG signal peptide. The N terminal methionine and/or signal peptide may be linked to the fragment.

Fragments of a nucleic acid sequence SEQ ID NO:1 can be 100% identical to the full length except missing at least one nucleotide from the 5′ and/or 3′ end, in each case with or without sequences encoding signal peptides and/or a methionine at position 1. Fragments can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of full length coding sequence SEQ ID NO:1, excluding any heterologous signal peptide added. The fragment can, preferably, comprise a fragment that encodes a polypeptide that is 95% or more, 96% or more, 97% or more, 98% or more or 99% or more homologous to the antigen SEQ ID NO:2 and additionally optionally comprise sequence encoding an N terminal methionine or heterologous signal peptide which is not included when calculating percent homology Fragments can further comprise coding sequences for an N terminal methionine and/or a signal peptide such as an immunoglobulin signal peptide, for example an IgE or IgG signal peptide. The coding sequence encoding the N terminal methionine and/or signal peptide may be linked to the fragment.

Fragments of SEQ ID NO:1 may comprise 30 or more nucleotides, including preferably sequences that encode an immunodominant epitope. In some embodiments, fragments of SEQ ID NO:1 may comprise 45 or more nucleotides, including preferably sequences that encode an immunodominant epitope. In some embodiments, fragments of SEQ ID NO:1 may comprise 60 or more nucleotides, including preferably sequences that encode an immunodominant epitope. In some embodiments, fragments of SEQ ID NO:1 may comprise 75 or more nucleotides, including preferably sequences that encode an immunodominant epitope. In some embodiments, fragments of SEQ ID NO:1 may comprise 90 or more nucleotides, including preferably sequences that encode an immunodominant epitope. In some embodiments, fragments of SEQ ID NO:1 may comprise 120 or more nucleotides, including preferably sequences that encode an immunodominant epitope. In some embodiments, fragments of SEQ ID NO:1 may comprise 150 or more nucleotides, including preferably sequences that encode an immunodominant epitope. In some embodiments, fragments of SEQ ID NO:1 may comprise 180 or more nucleotides, including preferably sequences that encode an immunodominant epitope. In some embodiments, fragments of SEQ ID NO:1 may comprise 210 or more nucleotides, including preferably sequences that encode an immunodominant epitope. In some embodiments, fragments of SEQ ID NO:1 may comprise 240 or more nucleotides, including preferably sequences that encode an immunodominant epitope. In some embodiments, fragments of SEQ ID NO:1 may comprise 270 or more nucleotides, including preferably sequences that encode an immunodominant epitope. In some embodiments, fragments of SEQ ID NO:1 may comprise 300 or more nucleotides, including preferably sequences that encode an immunodominant epitope. In some embodiments, fragments of SEQ ID NO:1 may comprise 360 or more nucleotides, including preferably sequences that encode an immunodominant epitope. In some embodiments, fragments of SEQ ID NO:1 may comprise 420 or more nucleotides, including preferably sequences that encode an immunodominant epitope. In some embodiments, fragments of SEQ ID NO:1 may comprise 480 or more nucleotides, including preferably sequences that encode an immunodominant epitope. In some embodiments, fragments of SEQ ID NO:1 may comprise 540 or more nucleotides, including preferably sequences that encode an immunodominant epitope. In some embodiments, fragments of SEQ ID NO:1 may comprise 600 or more nucleotides, including preferably sequences that encode an immunodominant epitope. In some embodiments, fragments of SEQ ID NO:1 may comprise 300 or more nucleotides, including preferably sequences that encode an immunodominant epitope. In some embodiments, fragments of SEQ ID NO:1 may comprise 660 or more nucleotides, including preferably sequences that encode an immunodominant epitope. In some embodiments, fragments of SEQ ID NO:1 may comprise 720 or more nucleotides, including preferably sequences that encode an immunodominant epitope. In some embodiments, fragments of SEQ ID NO:1 may comprise 780 or more nucleotides, including preferably sequences that encode an immunodominant epitope. In some embodiments, fragments of SEQ ID NO:1 may comprise coding sequences for the IgE leader sequences. In some embodiments, fragments of SEQ ID NO:1 do not comprise coding sequences for the IgE leader sequences. Fragments may comprise fewer than 60 nucleotides, in some embodiments fewer than 75 nucleotides, in some embodiments fewer than 90 nucleotides, in some embodiments fewer than 120 nucleotides, in some embodiments fewer than 150 nucleotides, in some embodiments fewer than 180 nucleotides, in some embodiments fewer than 210 nucleotides, in some embodiments fewer than 240 nucleotides, in some embodiments fewer than 270 nucleotides, in some embodiments fewer than 300 nucleotides, in some embodiments fewer than 360 nucleotides, in some embodiments fewer than 420 nucleotides, in some embodiments fewer than 480 nucleotides, in some embodiments fewer than 540 nucleotides, in some embodiments fewer than 600 nucleotides, in some embodiments fewer than 660 nucleotides, in some embodiments fewer than 720 nucleotides, and in some embodiments fewer than 780 nucleotides.

Fragments of SEQ ID NO:2 may comprise 15 or more amino acids, including preferably sequences that encode an immunodominant epitope. In some embodiments, fragments of SEQ ID NO:2 may comprise 18 or more amino acids, including preferably sequences that encode an immunodominant epitope. In some embodiments, fragments of SEQ ID NO:2 may comprise 21 or more amino acids, including preferably sequences that encode an immunodominant epitope. In some embodiments, fragments of SEQ ID NO:2 may comprise 24 or more amino acids, including preferably sequences that encode an immunodominant epitope. In some embodiments, fragments of SEQ ID NO:2 may comprise 30 or more amino acids, including preferably sequences that encode an immunodominant epitope. In some embodiments, fragments of SEQ ID NO:2 may comprise 36 or more amino acids, including preferably sequences that encode an immunodominant epitope. In some embodiments, fragments of SEQ ID NO:2 may comprise 42 or more amino acids, including preferably sequences that encode an immunodominant epitope. In some embodiments, fragments of SEQ ID NO:2 may comprise 48 or more amino acids, including preferably sequences that encode an immunodominant epitope. In some embodiments, fragments of SEQ ID NO:2 may comprise 54 or more amino acids, including preferably sequences that encode an immunodominant epitope. In some embodiments, fragments of SEQ ID NO:2 may comprise 60 or more amino acids, including preferably sequences that encode an immunodominant epitope. In some embodiments, fragments of SEQ ID NO:2 may comprise 18 or more amino acids, including preferably sequences that encode an immunodominant epitope. In some embodiments, fragments of SEQ ID NO:2 may comprise 72 or more amino acids, including preferably sequences that encode an immunodominant epitope. In some embodiments, fragments of SEQ ID NO:2 may comprise 90 or more amino acids, including preferably sequences that encode an immunodominant epitope. In some embodiments, fragments of SEQ ID NO:2 may comprise 120 or more amino acids, including preferably sequences that encode an immunodominant epitope. In some embodiments, fragments of SEQ ID NO:2 may comprise 150 or more amino acids, including preferably sequences that encode an immunodominant epitope. In some embodiments, fragments of SEQ ID NO:2 may comprise 180 or more amino acids, including preferably sequences that encode an immunodominant epitope. In some embodiments, fragments of SEQ ID NO:2 may comprise 210 or more amino acids, including preferably sequences that encode an immunodominant epitope. In some embodiments, fragments of SEQ ID NO:2 may comprise 240 or more amino acids, including preferably sequences that encode an immunodominant epitope. In some embodiments, fragments of SEQ ID NO:2 may comprise 260 or more amino acids, including preferably sequences that encode an immunodominant epitope. In some embodiments, fragments of SEQ ID NO:2 may comprise coding sequences for the IgE leader sequences. In some embodiments, fragments of SEQ ID NO:2 do not comprise coding sequences for the IgE leader sequences. Fragments may comprise fewer than 24 amino acids, in some embodiments fewer than 30 amino acids, in some embodiments fewer than 36 amino acids, in some embodiments fewer than 42 amino acids, in some embodiments fewer than 48 amino acids, in some embodiments fewer than 54 amino acids, in some embodiments fewer than 60 amino acids, in some embodiments fewer than 72 amino acids, in some embodiments fewer than 90 amino acids, in some embodiments fewer than 120 amino acids, in some embodiments fewer than 150 amino acids, in some embodiments fewer than 180 amino acids, in some embodiments fewer than 210 amino acids in some embodiments fewer than 240 amino acids, and in some embodiments fewer than 260 amino acids.

### HPV18 E6-E7 Fusion

Another aspect provides compositions comprising one or more nucleotide sequences encoding an HPV18 E6-E7 fusion antigen selected from the group consisting of: nucleotide sequence that encodes SEQ ID NO:10; a nucleotide sequence that is at least 95% homologous to a nucleotide sequence that encodes SEQ ID NO:10; a fragment of a nucleotide sequence that encodes SEQ ID NO:10; a nucleotide sequence that is at least 95% homologous to a fragment of a nucleotide sequence that encodes SEQ ID NO:10.

In some embodiments the compositions include HPV18 E6-E7 fusion antigens selected from the group consisting of: nucleotide sequence that encodes SEQ ID NO:10; a nucleotide sequence that is at least 95% homologous to a nucleotide sequence that encodes SEQ ID NO:10; a fragment of a nucleotide sequence that encodes SEQ ID NO:10; a nucleotide sequence that is at least 95% homologous to a fragment of a nucleotide sequence that encodes SEQ ID NO:10.

In another aspect of the invention, there are provided compositions comprising one or more nucleotide sequences encoding an HPV18 E6-E7 fusion antigen selected from the group consisting of: SEQ ID NO:9; a nucleotide sequence that is at least 95% homologous to SEQ ID NO:9; a fragment of SEQ ID NO:9; a nucleotide sequence that is at least 95% homologous to a fragment of SEQ ID NO:9.

In some embodiments the nucleotide sequences described herein is absent the leader sequence. In one embodiment, the nucleotide sequences comprising HPV18 E6-E7 fusion antigen is absent a leader sequence. In particular, the HPV18 E6-E7 fusion antigens including nucleotide sequence that encodes SEQ ID NO:10; are absent a leader sequence at 5′ end, for example nucleotide sequence encoding SEQ ID NO:12. In particular, the HPV16 E6-E7 fusion antigens including nucleotide sequence SEQ ID NO:9 are absent a leader sequence at 5′ end, for example nucleotide sequence comprising SEQ ID NO:11.

In some embodiments the compositions include HPV18 E6-E7 fusion antigens selected from the group consisting of: nucleotide sequence that encodes SEQ ID NO:14; a nucleotide sequence that is at least 95% homologous to a nucleotide sequence that encodes SEQ ID NO:14; a fragment of a nucleotide sequence that encodes SEQ ID NO:14; a nucleotide sequence that is at least 95% homologous to a fragment of a nucleotide sequence that encodes SEQ ID NO:14. SEQ ID NO:14 comprises the amino acid sequence of the HPV18 E6-E7 fusion antigen of SEQ ID NO:10 and further comprises an IgE leader sequence.

In another aspect of the invention, there are provided compositions comprising one or more nucleotide sequences encoding an HPV18 E6-E7 fusion antigen selected from the group consisting of: SEQ ID NO:13; a nucleotide sequence that is at least 95% homologous to SEQ ID NO:13; a fragment of SEQ ID NO:9; a nucleotide sequence that is at least 95% homologous to a fragment of SEQ ID NO:13. SEQ ID NO:13 comprises the nucleotide sequence of SEQ ID NO:9 encoding a HPV18 E6-E7 fusion antigen and further comprises a nucleotide sequence encoding an IgE leader sequence.

In some embodiments the nucleotide sequences of the present invention can be 80%, 81%, 82%, 83%, 84%, 85%, 86%, 87%, 88%, 89%, 90%, 91%, 92%, 93%, 94%, 95%, 96%, 97%, 98%, or 99% homologous with the provided nucleotide sequences; preferably 95%, 96%, 97%, 98%, or 99%; or 98% or 99%.

The nucleotide sequences provided can be included into one of a variety of known vectors or delivery systems, including a plasmid, viral vector, lipid vector, nanoparticle.; preferably a plasmid.

In additional aspects, provided are pharmaceutical compositions comprising the disclosed nucleotide sequences.

In some aspects, there are methods of inducing an effective immune response in an individual against more than one subtype of HPV thereby providing a prophylactic or therapeutic treatment against vulvar dysplasia, comprising administering to said individual a composition comprising one or more of the nucleotides sequences provided; preferably, the compositions have more than one antigen. The methods preferably include a step of introducing the provided nucleotide sequences into the individual by electroporation.

SEQ ID NO:9 comprises a nucleotide sequence that encodes a consensus immunogen of HPV18 E6 and E7 proteins. SEQ ID NO:13 includes SEQ ID NO:9 and further comprises an IgE leader sequence linked to the nucleotide sequence that encodes a consensus immunogen of HPV18 E6 and E7 proteins. SEQ ID NO:10 comprises the amino acid sequence for the consensus immunogen of HPV18 E6 and E7 proteins. SEQ ID NO:14 includes SEQ ID NO:10 and further comprises an IgE leader sequence linked to a consensus immunogen sequence. The IgE leader sequence is SEQ ID NO:12 and may be encoded by SEQ ID NO:11. SEQ ID NO:15 is the nucleic acid sequence of the plasmid pGX3002 with SEQ ID NO:13 incorporated for expression therein.

Further information regarding the HPV16 E6-E7 fusion antigen can be found at least in U.S. Pat. No. 8,389,706, which is incorporated by reference in its entirety.

In some embodiments, vaccines include SEQ ID NO:10, or a nucleic acid molecule that encodes SEQ ID NO:10. In some embodiments, vaccines include SEQ ID NO:9 as a nucleic acid molecule that encodes SEQ ID NO:10. In some embodiments, vaccines comprise SEQ ID NO:14 or a nucleic acid molecule that encodes SEQ ID NO:14. In some embodiments, vaccines comprise SEQ ID NO:13 as a nucleic acid molecule that encodes SEQ ID NO:14. In some embodiments, vaccines comprise SEQ ID NO:15.

Fragments of SEQ ID NO:10 or 14 may be 100% identical to the full length except missing at least one amino acid from the N and/or C terminal, in each case with or without signal peptides and/or a methionine at position 1. Fragments of SEQ ID NO:10 or 15 can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the full length SEQ ID NO:10 or 14, excluding any heterologous signal peptide added. The fragment can, preferably, comprise a fragment of SEQ ID NO:10 or 15 that is 95% or more, 96% or more, 97% or more, 98% or more or 99% or more homologous to SEQ ID NO:10 or 14 and additionally comprise an N terminal methionine or heterologous signal peptide which is not included when calculating percent homology. Fragments can further comprise an N terminal methionine and/or a signal peptide such as an immunoglobulin signal peptide, for example an IgE or IgG signal peptide. The N terminal methionine and/or signal peptide may be linked to the fragment.

Fragments of a nucleic acid sequence SEQ ID NO:9 or 13 can be 100% identical to the full length except missing at least one nucleotide from the 5′ and/or 3′ end, in each case with or without sequences encoding signal peptides and/or a methionine at position 1. Fragments can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of full length coding sequence SEQ ID NO:9 or 13, excluding any heterologous signal peptide added. The fragment can, preferably, comprise a fragment that encodes a polypeptide that is 95% or more, 96% or more, 97% or more, 98% or more or 99% or more homologous to the antigen SEQ ID NO:10 or 14 and additionally optionally comprise sequence encoding an N terminal methionine or heterologous signal peptide which is not included when calculating percent homology. Fragments can further comprise coding sequences for an N terminal methionine and/or a signal peptide such as an immunoglobulin signal peptide, for example an IgE or IgG signal peptide. The coding sequence encoding the N terminal methionine and/or signal peptide may be linked to the fragment.

Fragments of SEQ ID NO:9 may comprise 90 or more nucleotides. In some embodiments, fragments of SEQ ID NO:9 may comprise 180 or more nucleotides; in some embodiments, 270 or more nucleotides; in some embodiments 360 or more nucleotides; in some embodiments, 450 or more nucleotides; in some embodiments 540 or more nucleotides; in some embodiments, 630 or more nucleotides; in some embodiments, 720 or more nucleotides; and in some embodiments, 770 or more nucleotides. In some embodiments, fragments of SEQ ID NO:9 such as those set forth herein may further comprise coding sequences for the IgE leader sequences. In some embodiments, fragments of SEQ ID NO:9 do not comprise coding sequences for the IgE leader sequences. Fragments of SEQ ID NO:9 may comprise fewer than 180 nucleotides, in some embodiments fewer than 270 nucleotides, in some embodiments fewer than 360 nucleotides, in some embodiments fewer than 450 nucleotides, in some embodiments fewer than 540 nucleotides, in some embodiments fewer than 630 nucleotides, in some embodiments fewer than 690 nucleotides, in some embodiments fewer than 760 nucleotides, and in some embodiments fewer than 780 nucleotides.

Fragments of SEQ ID NO:10 may comprise 30 or more amino acids. In some embodiments, fragments of SEQ ID NO:10 may comprise 60 or more amino acids; in some embodiments, 90 or more amino acids; in some embodiments, 120 or more amino acids; in some embodiments; 150 or more amino acids; in some embodiments 180 or more amino acids; in some embodiments, 210 or more amino acids; and in some embodiments, 240 or more amino acids. Fragments may comprise fewer than 90 amino acids, in some embodiments fewer than 120 amino acids, in some embodiments fewer than 150 amino acids, in some embodiments fewer than 180 amino acids, in some embodiments fewer than 210 amino acids, and in some embodiments fewer than 240 amino acids.

All fragments of SEQ ID NO:13 comprise coding sequences encoding HPV sequences, i.e. the fragments of SEQ ID NO:13 must comprise sequences in addition to those encoding the IgE leader peptide. In some embodiments, fragments of SEQ ID NO:13 comprise 90 or more nucleotides. In some embodiments, fragments of SEQ ID NO:13 may comprise 180 or more nucleotides; in some embodiments, 270 or more nucleotides; in some embodiments 360 or more nucleotides; in some embodiments, 450 or more nucleotides; in some embodiments 540 or more nucleotides; in some embodiments, 630 or more nucleotides; in some embodiments, 720 or more nucleotides; in some embodiments, 810 or more nucleotides; and in some embodiments, 830 or more nucleotides. Fragments of SEQ ID NO:13 may comprise fewer than 180 nucleotides, in some embodiments fewer than 270 nucleotides, in some embodiments fewer than 360 nucleotides, in some embodiments fewer than 450 nucleotides, in some embodiments fewer than 540 nucleotides, in some embodiments fewer than 630 nucleotides, in some embodiments fewer than 690 nucleotides, in some embodiments fewer than 720 nucleotides, in some embodiments fewer than 780 nucleotides, and in some embodiments fewer than 840 nucleotides.

Fragments of SEQ ID NO:14 may comprise 30 or more amino acids including HPV sequences. In some embodiments, fragments of SEQ ID NO:14 may comprise 60 or more amino acids including HPV sequences; in some embodiments, 90 or more amino acids including HPV sequences; in some embodiments, 120 or more amino acids including HPV sequences; in some embodiments; 150 or more amino acids including HPV sequences; in some embodiments 180 or more amino acids including HPV sequences; in some embodiments, 210 or more amino acids including HPV sequences; in some embodiments, 240 or more amino acids including HPV sequences; and in some embodiments, 270 or more amino acids including HPV sequences. Fragments may comprise fewer than 90 amino acids including HPV sequences, in some embodiments fewer than 120 amino acids including HPV sequences, in some embodiments fewer than 150 amino acids including HPV sequences, in some embodiments fewer than 180 amino acids including HPV sequences, in some embodiments fewer than 210 amino acids including HPV sequences, in some embodiments fewer than 240 amino acids including HPV sequences, and in some embodiments fewer than 270 amino acids including HPV sequences.

In one embodiment, the HPV16 E6-E7 immunogen, HPV18 E6-E7 immunogen; or nucleic acid molecule encoding the HPV16 E6-E7 immunogen or HPV16 E6-E7 immunogen is administered in combination with IL-12. In one embodiment, IL-12 is encoded from a synthetic DNA plasmid.

Methods of treating or preventing vulvar dysplasia in a subject by inducing an immune response in an individual against HPV comprising administering to said individual a composition comprising a nucleic acid sequences provided herein. In some embodiments, the methods also include introducing the nucleic acid sequences into the individual by electroporation.

In some aspects, there are methods of treating or preventing vulvar dysplasia in a subject by inducing an immune response in an individual against HPV comprising administering to said individual a composition comprising a amino acid sequence provided herein. In some embodiments, the methods also include introducing the amino acid sequences into the individual by electroporation.

Improved vaccines comprise proteins and genetic constructs that encode proteins with epitopes that make them particularly effective as immunogens against which anti-HPV immune responses can be induced. Accordingly, vaccines can be provided to induce a therapeutic or prophylactic immune response. In some embodiments, the means to deliver the immunogen is a DNA vaccine, a recombinant vaccine, a protein subunit vaccine, a composition comprising the immunogen, an attenuated vaccine or a killed vaccine. In some embodiments, the vaccine comprises a combination selected from the groups consisting of: one or more DNA vaccines, one or more recombinant vaccines, one or more protein subunit vaccines, one or more compositions comprising the immunogen, one or more attenuated vaccines and one or more killed vaccines.

Aspects of the invention provide methods of delivering the coding sequences of the protein on nucleic acid molecule such as plasmid, as part of recombinant vaccines and as part of attenuated vaccines, as isolated proteins or proteins part of a vector.

According to some aspects of the present invention, compositions and methods are provided which prophylactically and/or therapeutically immunize an individual.

DNA vaccines are described in US. Pat. Nos. 5,593,972, 5,739,118, 5,817,637, 5,830,876, 5,962,428, 5,981,505, 5,580,859, 5,703,055, 5,676,594, and the priority applications cited therein, which are each incorporated herein by reference. In addition to the delivery protocols described in those applications, alternative methods of delivering DNA are described in US. Pat. Nos. 4,945,050 and 5,036,006, which are both incorporated herein by reference.

The present invention relates to improved attenuated live vaccines, improved killed vaccines and improved vaccines that use recombinant vectors to deliver foreign genes that encode antigens and well as subunit and glycoprotein vaccines. Examples of attenuated live vaccines, those using recombinant vectors to deliver foreign antigens, subunit vaccines and glycoprotein vaccines are described in U.S. Pat. Nos.: 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,3 64; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference.

When taken up by a cell, the genetic construct(s) may remain present in the cell as a. functioning extrachromosomal molecule and/or integrate into the cell’s chromosomal DNA. DNA may be introduced into cells where it remains as separate genetic material in the form of a plasmid or plasmids. Alternatively, linear DNA that can integrate into the chromosome may be introduced into the cell. When introducing DNA into the cell, reagents that promote DNA integration into chromosomes may be added. DNA sequences that are useful to promote integration may also be included in the DNA molecule. Alternatively, RNA may be administered to the cell. It is also contemplated to provide the genetic construct as a linear minichromosome including a centromere, telomeres and an origin of replication. Gene constructs may remain part of the genetic material in attenuated live microorganisms or recombinant microbial vectors which live in cells. Gene constructs may be part of genomes of recombinant viral vaccines where the genetic material either integrates into the chromosome of the cell or remains extrachromosomal. Genetic constructs include regulatory elements necessary for gene expression of a nucleic acid molecule. The elements include: a promoter, an initiation codon, a stop codon, and a polyadenylation signal. In addition, enhancers are often required for gene expression of the sequence that encodes the target protein or the immunomodulating protein. It is necessary that these elements be operable linked to the sequence that encodes the desired proteins and that the regulatory elements are operably in the individual to whom they are administered.

Initiation codons and stop codon are generally considered to be part of a nucleotide sequence that encodes the desired protein. However, it is necessary that these elements are functional in the individual to whom the gene construct is administered. The initiation and termination codons must be in frame with the coding sequence.

Promoters and polyadenylation signals used must be functional within the cells of the individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (MV) such as the BIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein.

Examples of polyadenylation signals useful to practice the present invention, especially in the production of a genetic vaccine for humans, include but are not limited to SV40 polyadenylation signals and LTR polyadenylation signals. In particular, the SV40 polyadenylation signal that is in pCEP4 plasmid (Invitrogen, San Diego CA), referred to as the SV40 polyadenylation signal, is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with mammalian origin of replication in order to maintain the construct extrachromosomally and produce multiple copies of the construct in the cell. Plasmids pVAX1, pCEP4 and pREP4 from Invitrogen (San Diego, CA) contain the Epstein Barr virus origin of replication and nuclear antigen EBNA-1 coding region which produces high copy episomal replication without integration.

In some preferred embodiments related to immunization applications, nucleic acid molecule(s) are delivered which include nucleotide sequences that encode protein of the invention, and, additionally, genes for proteins which further enhance the immune response against such target proteins. Examples of such genes are those which encode other cytokines and lymphokines such as alpha-interferon, gamma-interferon, platelet derived growth factor (PDGF), TNFα, TNFβ, GM-CSF, epidermal growth factor (EGF), IL-1, IL-2, IL-4, IL-5, IL-6, IL-10, IL-12, IL-18, MHC, CD80,CD86 and IL- 15 including IL-15 having the signal sequence deleted and optionally including the signal peptide from IgE. Other genes which may be useful include those encoding: MCP-1, MIP-la, MIP-1p, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof

An additional element may be added which serves as a target for cell destruction if it is desirable to eliminate cells receiving the genetic construct for any reason. A herpes thymidine kinase (tk) gene in an expressible form can be included in the genetic construct. The drug gangcyclovir can be administered to the individual and that drug will cause the selective killing of any cell producing tk, thus, providing the means for the selective destruction of cells with the genetic construct.

In order to maximize protein production, regulatory sequences may be selected which are well suited for gene expression in the cells the construct is administered into. Moreover, codons may be selected which are most efficiently transcribed in the cell. One having ordinary skill in the art can produce DNA constructs that are functional in the cells.

In some embodiments, gene constructs may be provided in which the coding sequences for the proteins described herein are linked to IgE signal peptide. In some embodiments, proteins described herein are linked to IgE signal peptide.

In some embodiments for which protein is used, for example, one having ordinary skill in the art can, using well known techniques, produce and isolate proteins of the invention using well known techniques. In some embodiments for which protein is used, for example, one having ordinary skill in the art can, using well known techniques, inserts DNA molecules that encode a protein of the invention into a commercially available expression vector for use in well known expression systems. For example, the commercially available plasmid pSE420 (Invitrogen, San Diego, Calif.) may be used for production of protein in E. coli. The commercially available plasmid pYES2 (Invitrogen, San Diego, Calif.) may, for example, be used for production in S. cerevisiae strains of yeast. The commercially available MAXBAC™ complete baculovirus expression system (Invitrogen, San Diego, Calif.) may, for example, be used for production in insect cells. The commercially available plasmid pcDNA I or pcDNA3 (Invitrogen, San Diego, Calif.) may, for example, be used for production in mammalian cells such as Chinese Hamster Ovary cells. One having ordinary skill in the art can use these commercial expression vectors and systems or others to produce protein by routine techniques and readily available starting materials. (See e.g., Sambrook et al., Molecular Cloning a Laboratory Manual, Second Ed. Cold Spring Harbor Press (1989) which is incorporated herein by reference.) Thus, the desired proteins can be prepared in both prokaryotic and eukaryotic systems, resulting in a spectrum of processed forms of the protein.

One having ordinary skill in the art may use other commercially available expression vectors and systems or produce vectors using well known methods and readily available starting materials. Expression systems containing the requisite control sequences, such as promoters and polyadenylation signals, and preferably enhancers are readily available and known in the art for a variety of hosts. See e.g., Sambrook et al., Molecular Cloning a Laboratory Manual, Second Ed. Cold Spring Harbor Press (1989). Genetic constructs include the protein coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes protein of the invention from readily available starting materials. The expression vector including the DNA that encodes the protein is used to transform the compatible host which is then cultured and maintained under conditions wherein expression of the foreign DNA takes place.

The protein produced is recovered from the culture, either by lysing the cells or from the culture medium as appropriate and known to those in the art. One having ordinary skill in the art can, using well known techniques, isolate protein that is produced using such expression systems. The methods of purifying protein from natural sources using antibodies which specifically bind to a specific protein as described above may be equally applied to purifying protein produced by recombinant DNA methodology.

In addition to producing proteins by recombinant techniques, automated peptide synthesizers may also be employed to produce isolated, essentially pure protein. Such techniques are well known to those having ordinary skill in the art and are useful if derivatives which have substitutions not provided for in DNA-encoded protein production.

The nucleic acid molecules may be delivered using any of several well known technologies including DNA injection (also referred to as DNA vaccination), recombinant vectors such as recombinant adenovirus, recombinant adenovirus associated virus and recombinant vaccinia.

Routes of administration include, but are not limited to, intramuscular, intransally, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. Preferred routes of administration include intramuscular, intraperitoneal, intradermal and subcutaneous injection. Genetic constructs may be administered by means including, but not limited to, electroporation methods and devices, traditional syringes, needleless injection devices, or “microprojectile bombardment gone guns”.

Examples of electroporation devices and electroporation methods preferred for facilitating delivery of the DNA vaccines, include those described in U.S. Pat. No. 7,245,963 by Draghia-Akli, et al., U.S. Pat. Pub. 2005/0052630 submitted by Smith, et al., the contents of which are hereby incorporated by reference in their entirety. Also preferred, are electroporation devices and electroporation methods for facilitating delivery of the DNA vaccines provided in co-pending and co-owned U.S. Pat. Application, Serial No. 11/874072, filed Oct. 17, 2007, which claims the benefit under 35 USC 119(e) to U.S. Provisional Applications Ser. Nos. 60/852,149, filed Oct. 17, 2006, and 60/978,982, filed Oct. 10, 2007, all of which are hereby incorporated in their entirety.

The following is an example of an embodiment using electroporation technology, and is discussed in more detail in the patent references discussed above: electroporation devices can be configured to deliver to a desired tissue of a mammal a pulse of energy producing a constant current similar to a preset current input by a user. The electroporation device comprises an electroporation component and an electrode assembly or handle assembly. The electroporation component can include and incorporate one or more of the various elements of the electroporation devices, including: controller, current waveform generator, impedance tester, waveform logger, input element, status reporting element, communication port, memory component, power source, and power switch. The electroporation component can function as one element of the electroporation devices, and the other elements are separate elements (or components) in communication with the electroporation component. In some embodiments, the electroporation component can function as more than one element of the electroporation devices, which can be in communication with still other elements of the electroporation devices separate from the electroporation component. The use of electroporation technology to deliver the improved HPV vaccine is not limited by the elements of the electroporation devices existing as parts of one electromechanical or mechanical device, as the elements can function as one device or as separate elements in communication with one another. The electroporation component is capable of delivering the pulse of energy that produces the constant current in the desired tissue, and includes a feedback mechanism. The electrode assembly includes an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism can receive the measured impedance and can adjust the pulse of energy delivered by the electroporation component to maintain the constant current.

In some embodiments, the plurality of electrodes can deliver the pulse of energy in a decentralized pattern. In some embodiments, the plurality of electrodes can deliver the pulse of energy in the decentralized pattern through the control of the electrodes under a programmed sequence, and the programmed sequence is input by a user to the electroporation component. In some embodiments, the programmed sequence comprises a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

In some embodiments, the feedback mechanism is performed by either hardware or software. Preferably, the feedback mechanism is performed by an analog closed-loop circuit. Preferably, this feedback occurs every 50 µs, 20 µs, 10 µs or 1 µs, but is preferably a real-time feedback or instantaneous (i.e., substantially instantaneous as determined by available techniques for determining response time). In some embodiments, the neutral electrode measures the impedance in the desired tissue and communicates the impedance to the feedback mechanism, and the feedback mechanism responds to the impedance and adjusts the pulse of energy to maintain the constant current at a value similar to the preset current. In some embodiments, the feedback mechanism maintains the constant current continuously and instantaneously during the delivery of the pulse of energy.

In some embodiments, the nucleic acid molecule is delivered to the cells in conjunction with administration of a polynucleotide function enhancer or a genetic vaccine facilitator agent. Polynucleotide function enhancers are described in U.S. Serial No. 5,593,972, 5,962,428 and International Application Serial Number PCT/US94/00899 filed Jan. 26, 1994, which are each incorporated herein by reference. Genetic vaccine facilitator agents are described in U.S. Serial No. 021,579 filed Apr. 1, 1994, which is incorporated herein by reference. The co-agents that are administered in conjunction with nucleic acid molecules may be administered as a mixture with the nucleic acid molecule or administered separately simultaneously, before or after administration of nucleic acid molecules. In addition, other agents which may function transfecting agents and/or replicating agents and/or inflammatory agents and which may be co-administered with a GVF include growth factors, cytokines and lymphokines such as α-interferon, gamma-interferon, GM-CSF, platelet derived growth factor (PDGF), TNF, epidermal growth factor (EGF), IL-1, IL-2, IL-4, IL-6, IL-10, IL-12 and IL-15 as well as fibroblast growth factor, surface active agents such as immune-stimulating complexes (ISCOMS), Freunds incomplete adjuvant, LPS analog including monophosphoryl Lipid A (WL), muramyl peptides, quinone analogs and vesicles such as squalene and squalene, and hyaluronic acid may also be used administered in conjunction with the genetic construct In some embodiments, an immunomodulating protein may be used as a GVF. In some embodiments, the nucleic acid molecule is provided in association with PLG to enhance delivery/uptake.

The pharmaceutical compositions according to the present invention comprise about 1 nanogram to about 2000 micrograms of DNA. In some preferred embodiments, pharmaceutical compositions according to the present invention comprise about 5 nanogram to about 1000 micrograms of DNA. In some preferred embodiments, the pharmaceutical compositions contain about 10 nanograms to about 800 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms of DNA. In some preferred embodiments, the pharmaceutical compositions contain about 100 to about 200 microgram DNA.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

According to some embodiments of the invention, methods of inducing immune responses are provided. The vaccine may be a protein based, live attenuated vaccine, a cell vaccine, a recombinant vaccine or a nucleic acid or DNA vaccine. In some embodiments, methods of inducing an immune response in individuals against an immunogen, including methods of inducing mucosal immune responses, comprise administering to the individual one or more of CTACK protein, TECK protein, MEC protein and functional fragments thereof or expressible coding sequences thereof in combination with an isolated nucleic acid molecule that encodes protein of the invention and/or a recombinant vaccine that encodes protein of the invention and/or a subunit vaccine that protein of the invention and/or a live attenuated vaccine and/or a killed vaccine. The one or more of CTACK protein, TECK protein, MEC protein and functional fragments thereof may be administered prior to, simultaneously with or after administration of the isolated nucleic acid molecule that encodes an immunogen; and/or recombinant vaccine that encodes an immunogen and/or subunit vaccine that comprises an immunogen and/or live attenuated vaccine and/or killed vaccine. In some embodiments, an isolated nucleic acid molecule that encodes one or more proteins of selected from the group consisting of: CTACK, TECK, MEC and functional fragments thereof is administered to the individual.

The present invention is further illustrated in the following Example. It should be understood that this Example, while indicating embodiments of the invention, is given by way of illustration only. From the above discussion and this Example, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

Each of the U.S. Patents, U.S. Applications, and references cited throughout this disclosure are hereby incorporated in their entirety by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

The management of vulvar dysplasia, also known as vulvar high-grade squamous intraepithelial lesions (HSIL) remains challenging. Surgical treatments are disfiguring and have a high recurrence rate (up to 34% at 6 months) post-treatment (Frega et al., The reinfection rate of high-risk HPV and the recurrence rate of vulvar intraepithelial neoplasia (VIN) usual type after surgical treatment. Med Sci Monit. 2011;17(9):CR532-CR535.DOI:10.12659/msm.881941).

Less than 1 in 20 women with vulvar HSIL exhibit spontaneous resolution, and without adequate treatment, will progress to vulvar cancer which is estimated to lead to the death of approximately 1350 women in the US in 2020 (cancer.org/cancer/vulvarcancer/about/key-statistics.html).

Presented herein is the first report of efficacy and safety of VGX-3100, a DNA-based HPV-16/18-specific immunotherapy, in a population with HPV-16/18-associated vulvar HSIL. Reports on 12 of the 22 women who have completed their efficacy assessment 6 months following treatment with VGX-3100 & Electroporation (EP) are generated.

HPV-201 is a Phase 2, open-label efficacy study of VGX-3100 administered by IM injection followed by EP in adult women with histologically confirmed vulvar HSIL associated with HPV-16 and/or HPV-18. VGX-3100 is a refrigerated formulation comprised of two DNA plasmids encoding E6 and E7 proteins of HPV types 16 and 18 (Hollenberg, RK, et al. (2019) Safety and immunogenicity of VGX-3100 formulations in a healthy young adult population, Human Vaccines & Immunotherapeutics, DOI: 10.1080/21645515.2019.1695459). The aim of these studies is to use VGX-3100 is to treat HPV-16/18 positive HSIL. 33 women with tissue-confirmed HPV-16/18-related vulvar HSIL, received (2:1) VGX-3100 intramuscularly with Electroporation at 0, 1, 3, and 6 months (4 doses), or VGX-3100 with EP (4 doses) and topical imiquimod thrice weekly for 20 weeks (VGX-3100/IMQ). The efficacy assessment was proportion of subjects without vulvar HSIL and non-detectability of HPV-16/18 (by SPF-10) in vulvar tissue post-treatment. Efficacy endpoints included regression of HSIL, non-detectability of HPV16/18, and lesion size reduction.

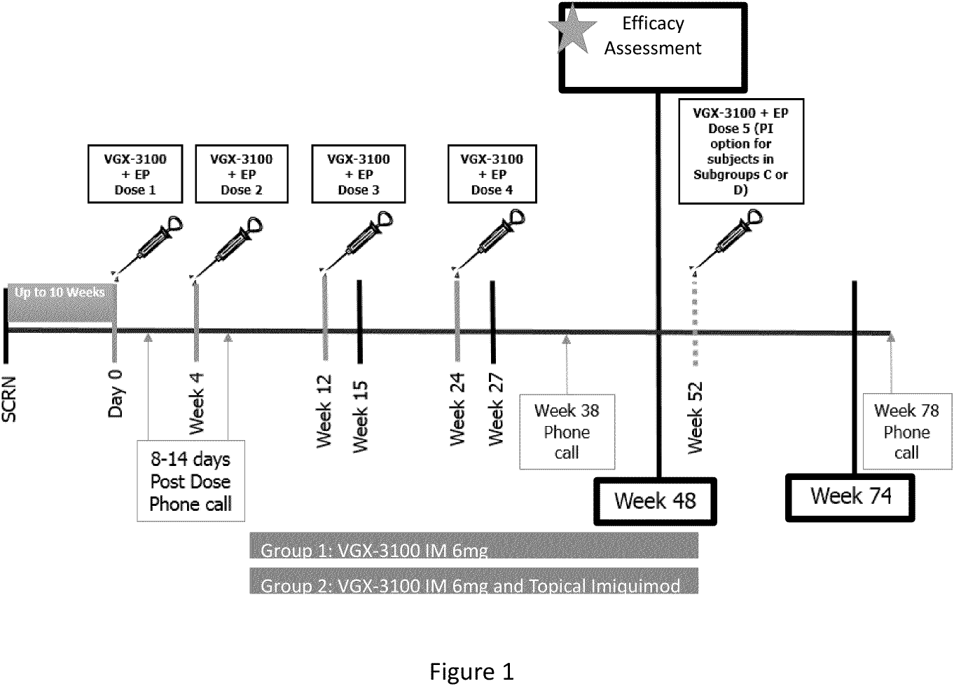

Adult female subjects were selected to participate in this study based on eligibility criteria which included histological confirmation of HSIL based on screening vulvar biopsy samples which were read by 2 independent pathologists, and tissue genotyping to confirm presence of HPV-16 and or HPV-18. The study design is shown in FIG. 1

The VGX-3100 drug is a refrigerated formulation comprised of two DNA plasmids which encode the E6 and E7 proteins of HPV subtypes 16 and 18. Four doses of VGX-3100 at 6 miligrams per dose, were delivered intramuscularly in the subject’s deltoid or quadriceps, followed by electroporation with the CELLECTRA 2000 device. These four doses occurred at the subjects’ Day 0, Week 4, Week 12 and Week 24 visits, with a potential additional dose available at Week 52 to partial treatment responders who were identified based on histologic and lesion area changes. This treatment was given alone or in combination with topical imiquimod, which was administered 3 times per week by the subject at home, starting from Day 0 and continued through Week 20.

At Week 48, the subject’s vulvar biopsy or excisional samples were obtained and again evaluated for histology and virology. Based on these results, at Week 52 subjects may 1) continue on study with standard of care, for those considered responders, 2) receive an additional treatment dose which is indicated as optional Dose 5, for those considered partial responders or 3) receive excisional treatment for those considered non-responders to study treatment.

Following this, all subjects continue on follow up for approximately 6 months through Week 78, which is the last visit of the study. Throughout the study visits, vulvar photography was performed and resulting photographs were used to obtain lesion area measurements.

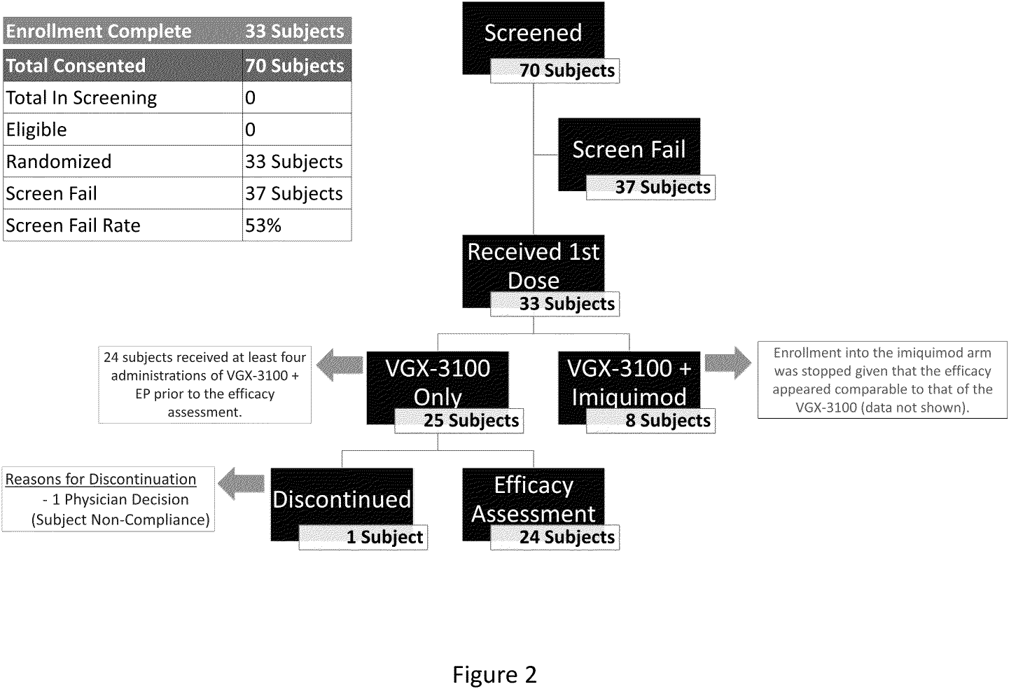

To enroll this study, 70 subjects were screened, and of whom 37 subjects screen failed at a rate of 53 percent (FIG. 2). 33 women meeting enrollment criteria were randomized 2 to 1, leading 25 subjects to receive VGX-3100 with EP, and 8 subjects to receive VGX-3100 with EP and topical imiquimod (FIG. 2). Of these 25 subjects, 1 subject was withdrawn due to physician’s decision and 24 subjects received all four administrations of VGX-3100 and EP, meaning the subjects have completed the study through Week 48.

While all subjects have since completed their last study visits as of December 2020, this report is focused on the 24 women who have completed their efficacy assessment at Week 48, which is 6 months following treatment with VGX-3100 & Electroporation (EP) alone

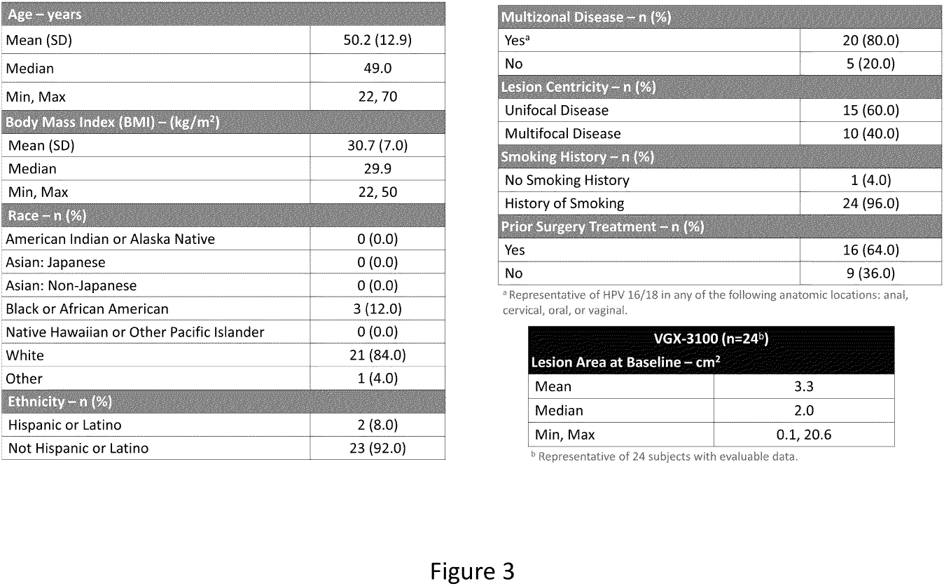

FIG. 3 shows the subjects; demographics data at baseline, as collected at screening. Of the 24 female subjects who received VGX-3100 & electroporation, the average age was 50.2 years, and a median age of 49 years was observed. Regarding subject body mass index, the average was 30.7, with a median of 29.9. 84.0% of our subjects identified as White, and Not Hispanic or Latino, 12% of subjects identified as Black or African American, and the remaining 4% of subjects identifying as Other, and Hispanic or Latino.

One of the subjects had no prior history of smoking, with the remaining 24 subjects self-reporting a prior or current history of smoking.

Five of the 25 subjects did not have observed multizonal disease, while twenty subjects experienced multizonal disease involving the anal, cervical, oral and vaginal locations.

Consistent with the literature, 64 percent of the subjects had received prior surgical treatment, and the remaining 36 percent did not have any prior treatment surgeries.

At baseline, 60 percent of subjects (15) had unifocal disease, and 40 percent of subjects (10) had multifocal disease. In the case the subject had multifocal disease, the two lesions that potentially contained the most advanced disease, as judged by the principal investigator at baseline, were selected for biopsy and the results were used to determine eligibility. Any new lesions or existing lesions suspicious for progression were biopsied at any appropriate point.

The baseline photography data was evaluable for 24 these subjects, and indicated an average lesion area of 3.3 cm squared, with a median of 2.0 cm squared (range of 0.1 to 20.6 cm squared).

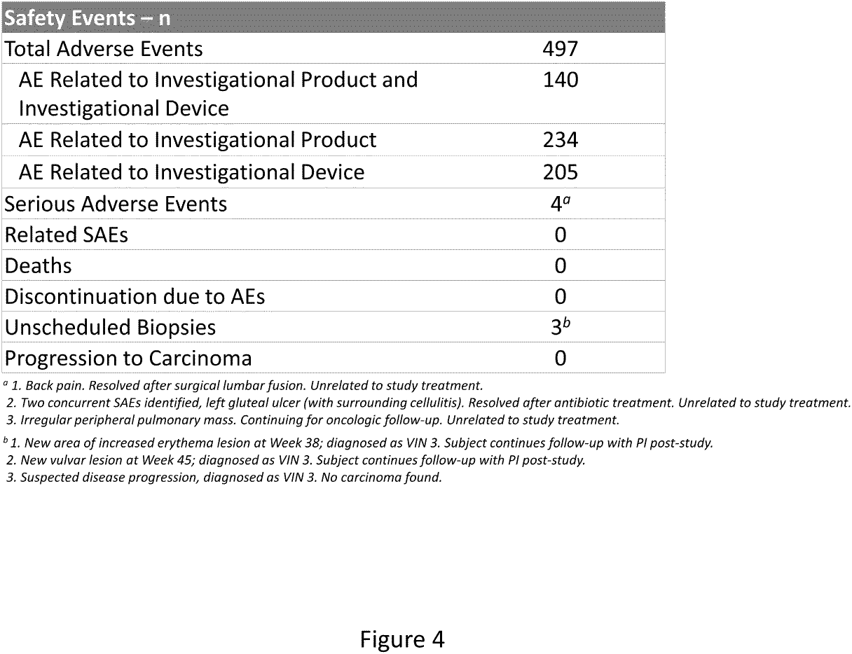

FIG. 4 shows the safety events in the study. Four hundred and ninety seven total adverse events were reported, with the relatedness to investigational product and investigational device reported below. There were 4 serious adverse events reported, all unrelated to study treatment. One subject reported severe back pain, and outside of the context of the study, this subject received surgical lumbar fusion which resolved her pain. Another subject experienced a gluteal ulcer with surrounding cellulitis, which were both resolved after a visit to an emergency room. Finally, one subject had an irregular peripheral pulmonary mass identified during a routine screening. This subject has continued to follow up and to date there has been no confirmation of carcinoma.

There have been 3 unscheduled biopsies, in which no carcinoma was found. All of these subjects continued on study for follow-up and completed the trial.

There have been no related serious adverse events, no deaths, no progression to carcinoma and no discontinuations due to adverse events.

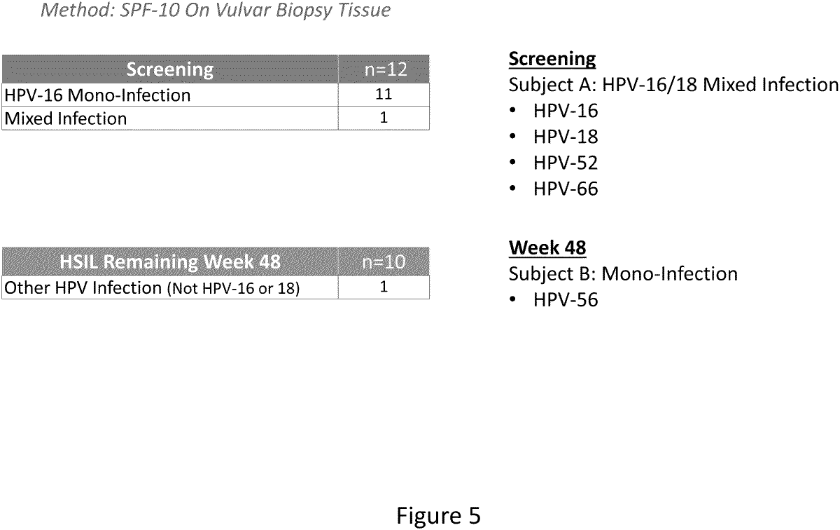

Subjects who enrolled in the study were required to have confirmed presence of HPV-16 and or HPV-18 based on tissue genotyping, which was performed using SPF-10 assay. Of 12 subjects evaluated within the VGX-3100 group, 11 had HPV-16 mono-infection, with the remaining 1 subject having a mixed infection which was comprised of HPV-16, HPV-18, HPV-52 and HPV-66, all in one tissue biopsy sample (FIG. 5).

Of this same set of subjects, 10 had HSIL remaining at the endpoint assessment at Week 48. Nine of the subjects with remaining HSIL had a remaining HPV-16 infection. However, only one subject with remaining HSIL had a non-HPV-16 or 18 infection, which was a mono-infection of HPV-56 (FIG. 5).

At 6 months following treatment, three out of twenty subjects (15%) experienced resolution of their HPV-16 and/or HPV-18 as well as their HSIL. Of this, two subjects (10%) had their lesion heal completely, as their lesions regressed to normal with no evidence of LSIL.

Of women with evaluable lesion area results, twelve of nineteen evaluable subjects had either a partial or complete reduction in their lesion size, meaning their lesion decreased by 25% or more. This corresponds to an average decrease of 1.68 cm squared and median of 1.36 cm squared. The range of decrease was a minimum decrease of 0.002 cm squared, and the largest decrease was 6.76 cm squared.

In summary, these final efficacy results from 6 months post-treatment indicates that VGX-3100 has a therapeutic effect upon HPV-16 and or HPV-18-associated vulvar HSIL. Given the high medical need and current limitations of surgery, an immuno-therapeutic approach would represent a significant advancement in the management of vulvar HSIL. Complete trial data will be based upon all enrolled subjects who have reached efficacy assessment through, and those within follow up through 18 months after the fourth treatment dose

1. A method for treating or preventing vulvar dysplasia in an individual comprising administering to the individual a composition comprising at least one nucleic acid molecule encoding at least one selected from the group consisting of: an HPV16 antigen and an HPV18 antigen.

2. The method of claim 1, wherein the HPV16 antigen is an HPV16 E6-E7 fusion antigen.

3. The method of claim 1, wherein the HPV18 antigen is an HPV18 E6-E7 fusion antigen.

4. The method of claim 1, wherein the composition comprises a nucleotide sequence encoding an HPV16 antigen and a nucleotide sequence encoding an HPV18 antigen.

5. The method of claim 2, wherein the nucleic acid molecule comprises one or more nucleotide sequences selected from the group consisting of:
a nucleotide sequence that encodes SEQ ID NO:2;
a nucleotide sequence that is at least 95% homologous to a nucleotide sequence that encodes SEQ ID NO:2;
a fragment of a nucleotide sequence that encodes SEQ ID NO:2;
a nucleotide sequence that is at least 95% homologous to a fragment of a nucleotide sequence that encodes SEQ ID NO:2.

6. The method of claim 5, wherein the nucleic acid molecule comprises a nucleotide sequence that is at least 98% homologous to a nucleotide sequence that encodes SEQ ID NO:2.

7. The method of claim 5, wherein the nucleic acid molecule comprises a nucleotide sequence that is at least 99% homologous to a nucleotide sequence that encodes SEQ ID NO:2.

8. The method of claim 5, where the nucleotide sequences encoding the HPV16 E6-E7 fusion antigen are without a leader sequence at 5′ end that is a nucleotide sequence that encodes SEQ ID NO:7.

9. The method of claim 2, wherein the nucleic acid molecule comprises one or more nucleotide sequences selected from the group consisting of:
a nucleotide sequence comprising SEQ ID NO:1;
a nucleotide sequence that is at least 95% homologous SEQ ID NO: 1;
a fragment of SEQ ID NO: 1;
a nucleotide sequence that is at least 95% homologous to a fragment of SEQ ID NO: 1.

10. The method of claim 9, wherein the nucleic acid molecule comprises a nucleotide sequence that is at least 98% homologous to SEQ ID NO: 1.

11. The method of claim 9, wherein the nucleic acid molecule comprises a nucleotide sequence that is at least 99% homologous to SEQ ID NO: 1.

12. The method of claim 3, wherein the nucleic acid molecule comprises one or more nucleotide sequences selected from the group consisting of:
a nucleotide sequence that encodes SEQ ID NO: 10;
a nucleotide sequence that is at least 95% homologous to a nucleotide sequence that encodes SEQ ID NO: 10;
a fragment of a nucleotide sequence that encodes SEQ ID NO: 10;
a nucleotide sequence that is at least 95% homologous to a fragment of a nucleotide sequence that encodes SEQ ID NO: 10.

13. The method of claim 12, wherein the nucleic acid molecule comprises a nucleotide sequence that is at least 98% homologous to a nucleotide sequence that encodes SEQ ID NO: 10.

14. The method of claim 12, wherein the nucleic acid molecule comprises a nucleotide sequence that is at least 99% homologous to a nucleotide sequence that encodes SEQ ID NO: 10.

15. The method of claim 12, where the nucleotide sequences encoding the HPV16 E6-E7 fusion antigen further comprises a nucleotide sequence encoding a leader sequence.

16. The method of claim 3, wherein the nucleic acid molecule comprises one or more nucleotide sequences selected from the group consisting of:
a nucleotide sequence comprising SEQ ID NO:9;
a nucleotide sequence that is at least 95% homologous SEQ ID NO:9;
a fragment of SEQ ID NO:9;
a nucleotide sequence that is at least 95% homologous to a fragment of SEQ ID NO:9.

17. The method of claim 16, wherein the nucleic acid molecule comprises a nucleotide sequence that is at least 98% homologous to SEQ ID NO:9.

18. The method of claim 16, wherein the nucleic acid molecule comprises a nucleotide sequence that is at least 99% homologous to SEQ ID NO:9.

19. The method of claim 1, wherein the at least one nucleic acid molecule comprises at least one plasmid.

20. The method of claim 1, wherein the composition is a pharmaceutical composition.

21. The method of claim 1, further comprising administering to the individual a composition comprising an adjuvant.

22. The method of claim 4, comprising administering to the individual a nucleotide sequence encoding an HPV16 E6-E7 fusion antigen and a nucleotide sequence encoding an HPV18 E6-E7 fusion antigen;
wherein the nucleotide sequence encoding the HPV16 E6-E7 fusion antigen is selected from the group consisting of: a nucleotide sequence that encodes SEQ ID NO:2; a nucleotide sequence that is at least 95% homologous to a nucleotide sequence that encodes SEQ ID NO:2; a fragment of a nucleotide sequence that encodes SEQ ID NO:2; and a nucleotide sequence that is at least 95% homologous to a fragment of a nucleotide sequence that encodes SEQ ID NO:2; and
wherein the nucleotide sequence encoding the HPV18 E6-E7 fusion antigen is selected from the group consisting of: a nucleotide sequence that encodes SEQ ID NO: 10; a nucleotide sequence that is at least 95% homologous to a nucleotide sequence that encodes SEQ ID NO: 10; a fragment of a nucleotide sequence that encodes SEQ ID NO: 10; and a nucleotide sequence that is at least 95% homologous to a fragment of a nucleotide sequence that encodes SEQ ID NO: 10 .

23. The method of claim 1, wherein administering said nucleic acid molecule to the individual comprises electroporation.
